(19) United States
(12) Patent Application Publication
(10) Pub. No.: US 2026/0021247 A1
(43) Pub. Date: Jan. 22, 2026

(54) INFUSION PUMP DRUG DIVERSION SECURITY SYSTEM

(71) Applicants: Baxter International Inc., Deerfield, IL (US); BAXTER HEALTHCARE SA, Glattpark (Opfikon) (CH)

(72) Inventors: Rajesh Swarnkar, Bangalore (IN); Sadhu Sharan Prasad, Chennai (IN); Vikasa N R, Bangalore (IN); Abhishek Alva, Mangalore (IN); Advitya Sharma, Bilaspur (IN); . Ranjeet, Bangalore (IN)

(21) Appl. No.: 19/264,460

(22) Filed: Jul. 9, 2025

(30) Foreign Application Priority Data
Jul. 19, 2024 (IN) 202441055349

(51) Int. Cl. A61M 5/14; E05B 65/00

(52) U.S. Cl. CPC A61M 5/1413; E05B 65/00; A61M 2005/1405; A61M 2205/14; A61M 2205/276

(57) ABSTRACT

A system and apparatus for securing a pump are disclosed. The system includes a patient-controlled analgesia (“PCA”) pump, a security door, a security frame assembly, and a diversion guard. The PCA pump can be placed in the security frame assembly. The security door interlocks with the security frame assembly and pivots about it from an open configuration to a closed configuration. When the security door is in a closed configuration, the medicament-administering components of the PCA pump are out of reach from unauthorized users. As an additional physical barrier, the diversion guard selectively couples to the security frame assembly, such that it blocks a physical gap between the security door and security frame assembly when the security door is in a closed configuration.

### PRIORITY CLAIM AND CROSS-REFERENCE TO RELATED APPLICATIONS

The present application claims priority to and the benefit of Indian Provisional Patent Application No. 202441055349 filed Jul. 19, 2024, which is incorporated herein by reference in its entirety and relied upon.

### BACKGROUND

Unauthorized tampering with medicament delivery systems, such as patient-controlled analgesia (“PCA”) pumps, to obtain pain medication, creates a risk to patient safety as well as inefficiencies in healthcare systems. Drug diversion by a malicious individual or clinician could result in a patient not receiving the necessary dose of medicament, which could be detrimental to the patient's outcome. Similarly, drug diversion can be costly and taxing on a health care system's finite resources. Finally, drug diversion can lead to misuse of medicaments like narcotic drugs, which can lead to life-threatening injuries or complications.

Current security systems trigger an alarm as soon as they detect potential unauthorized access to a pump. As an additional measure, current security systems include a security door assembly that encapsulates the pump, so as to prevent physical access and manipulation of the syringe holder. Users can lock the security door assembly as a way of preventing access to the syringe holder. However, unauthorized users can access syringe holders by tampering the security door assembly through the gap locations between the security door and the pump.

As a result, in current security systems, when the unauthorized user is able to make contact with the syringe holder, using external tools inserted through the gap location, medicament can be diverted. Therefore, a security system that includes a drug diversion guard that redundantly blocks the medicament delivery system from external tools by blocking gap locations is desirable.

### SUMMARY

Example systems, methods, and apparatus are disclosed herein for a system and method for securing a pump.

In light of the disclosure herein, and without limiting the scope of the invention in any way, in a first aspect of the present disclosure, which may be combined with any other aspect listed herein unless specified otherwise, a patient-controlled analgesia pump includes a pump body, a syringe holder, a security door, a security frame assembly, and a diversion guard.

In a second aspect of the present disclosure, which may be combined with any other aspect listed herein unless specified otherwise, the pump body is configured to couple to the security frame assembly, and the security door is configured to interlock with the pump body and security frame assembly such that the security door pivots relative to the security frame assembly.

In a third aspect of the present disclosure, which may be combined with any other aspect listed herein unless specified otherwise, the security door assembly can be oriented to a first position where the security door assembly pivots to a closed position, and the security door assembly can be oriented to a second position where the security door assembly pivots to an open position.

In a fourth aspect of the present disclosure, which may be combined with any other aspect listed herein unless specified otherwise, the syringe holder is not accessible when the security door is in a closed position.

In a fifth aspect of the present disclosure, which may be combined with any other aspect listed herein unless specified otherwise, the syringe holder is accessible when the security door is in an open position.

In a sixth aspect of the present disclosure, which may be combined with any other aspect listed herein unless specified otherwise, the diversion guard is configured to selectively couple to the security frame assembly.

In a seventh aspect of the present disclosure, which may be combined with any other aspect listed herein unless specified otherwise, when the diversion guard is selectively coupled to the security frame assembly and the security door is in a closed position, the diversion guard covers a space between the security door and the security frame assembly.

In an eight aspect of the present disclosure, which may be combined with any other aspect listed herein unless specified otherwise, the diversion guard is comprised of a strong and robust material, such that the diversion guard is breakproof.

In a ninth aspect of the present disclosure, which may be combined with any other aspect listed herein unless specified otherwise, the security frame assembly includes a deadbolt lock assembly configured to interact with a complimentary deadbolt receiver on the security door.

In a tenth aspect of the present disclosure, which may be combined with any other aspect listed herein unless specified otherwise, the deadbolt lock assembly and complimentary deadbolt receiver can be selectively locked together.

In an eleventh aspect of the present disclosure, which may be combined with any other aspect listed herein unless specified otherwise, one of the security door or the security frame assembly further includes at least one sensor.

In a twelfth aspect of the present disclosure, which may be combined with any other aspect listed herein unless specified otherwise, the at least one sensor is configured to detect the presence of at least one foreign object on the syringe holder when the security door is in a closed position.

In a thirteenth aspect of the present disclosure, which may be combined with any other aspect listed herein unless specified otherwise, a pump security system includes a patient-controlled analgesia pump, a security door, a security frame assembly, and a diversion guard. The patient-controlled analgesia pump includes a syringe holder.

In a fourteenth aspect of the present disclosure, which may be combined with any other aspect listed herein unless specified otherwise, the security door and security frame assembly are configured to enclose the patient-controlled analgesia pump, and the security door interlocks with the patient-controlled analgesia pump and security frame assembly such that the security door pivots relative to the security frame assembly.

In a fifteenth aspect of the present disclosure, which may be combined with any other aspect listed herein unless specified otherwise, the security door assembly can be oriented to a first position where the security door assembly pivots to a closed position, and the security door assembly can be oriented to a second position where the security door assembly pivots to an open position.

In a sixteenth aspect of the present disclosure, which may be combined with any other aspect listed herein unless specified otherwise, the diversion guard is configured to selectively couple to the security frame assembly, and when the diversion guard is selectively coupled to the security frame assembly and the security door is in a closed position, the diversion guard covers a space between the security door and the security frame assembly.

In a seventeenth aspect of the present disclosure, which may be combined with any other aspect listed herein unless specified otherwise, the diversion guard selectively couples to the patient-controlled analgesia pump assembly.

In an eighteenth aspect of the present disclosure, which may be combined with any other aspect listed herein unless specified otherwise, the diversion guard is comprised of a strong and robust material, such that the diversion guard is breakproof.

In a nineteenth aspect of the present disclosure, which may be combined with any other aspect listed herein unless specified otherwise, the diversion guard is configured to cover a space between a security door and a security frame assembly when the patient-controlled analgesia pump assembly is in a closed configuration.

In a twentieth aspect of the present disclosure, which may be combined with any other aspect listed herein unless specified otherwise, when the diversion guard covers the space between a security door and a security frame assembly when the patient-controlled analgesia pump assembly is in a closed configuration, an unauthorized user cannot access a syringe holder in the patient-controlled analgesia pump assembly.

In a twenty-first aspect of the present disclosure, any of the structure, functionality, and alternatives disclosed in connection with any one or more of FIGS. 1 to 8 may be combined with any other structure, functionality, and alternatives disclosed in connection with any other one or more of FIGS. 1 to 8.

In light of the present disclosure and the above aspects, it is therefore an advantage of the present disclosure to prevent physical access to pump components.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

Some example apparatus embodiments of the invention, and example procedures for making and using one or more example embodiments, are described in detail herein and by way of example, with reference to the accompanying drawings (which are not necessarily drawn to scale with regard to any internal or external structures shown) and in which like reference characters designate like elements throughout the several views, and in which:

FIGS. 1A-B illustrate perspective views of a current security door assembly during drug diversion, according to an example of the present disclosure.

FIGS. 2A-C illustrate bottom views of a current security door assembly, according to an example of the present disclosure.

FIG. 3 illustrates a perspective view of an infusion pump utilizing an infusion pump drug diversion guard, in the locked position, according to an example of the present disclosure.

FIG. 4 illustrates a perspective view of an infusion pump drug diversion guard, according to an example of the present disclosure.

FIG. 5 illustrates a front view of an infusion pump drug diversion guard, according to an example of the present disclosure.

FIG. 6 illustrates a top view of an infusion pump drug diversion guard, according to an example of the present disclosure.

FIG. 7 illustrates close-up perspective views of a corner of a security door assembly on an infusion pump with an infusion pump diversion guard installed, according to an example of the present disclosure.

FIG. 8 illustrates a close-up perspective view of a lock area of a security door assembly on an infusion pump with an infusion pump diversion guard installed, according to an example of the present disclosure.

### DETAILED DESCRIPTION OF EXAMPLE EMBODIMENTS

The present disclosure generally describes a pump security system and methods of using the same that guard against unauthorized access to pump components. Physical components such as locks, enclosure assemblies, and/or sensors may be used to physically prevent unauthorized access to pump components. Additionally, virtual security measures such as alarms may also be used to warn of unauthorized access to pump components. Both the physical and virtual security components may be used in a complimentary fashion. In an example, sensors may detect tampering with medicament delivery, which may trigger an alarm. In an additional example, diversion locks are disclosed that introduce physical redundancies that cover gaps between pump security doors and security frame assemblies.

### Current Security Door Assembly Drug Diversion

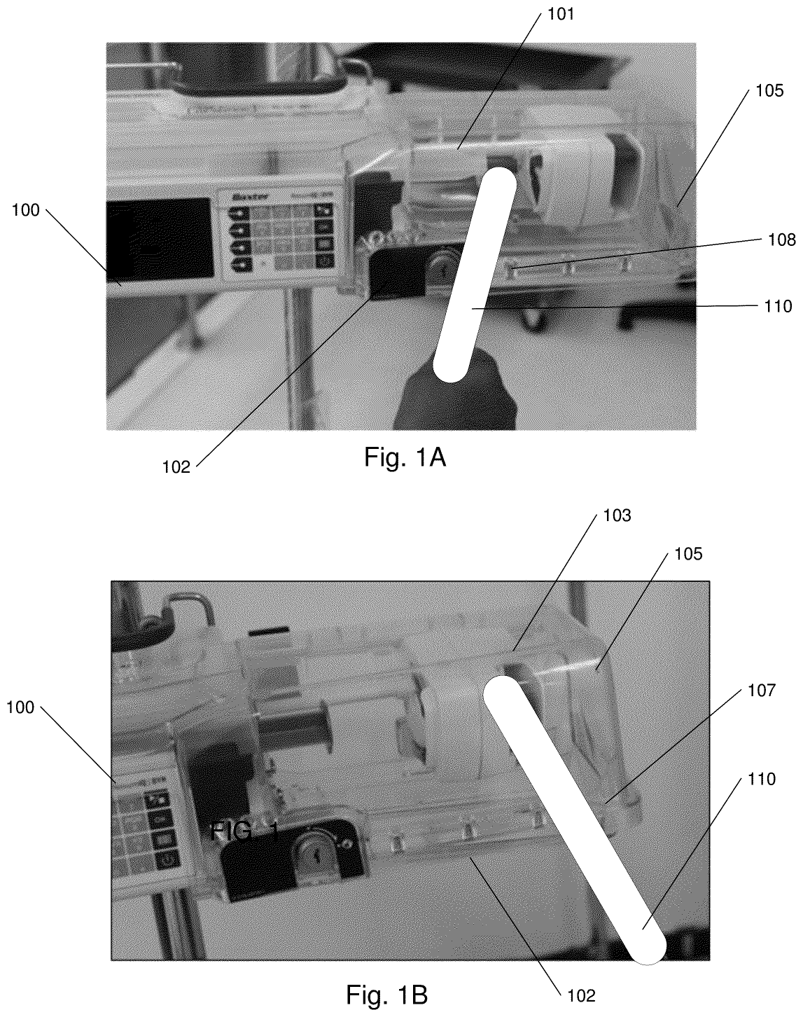

FIGS. 1A-B illustrate perspective views of a current security door assembly during drug diversion. Specifically, FIG. 1A shows an unauthorized user diverting medicament by inserting a tool through a security door assembly. As seen in FIG. 1A, in a current security door assembly, the unauthorized user can insert a tool 110 through a gap between a security frame assembly 102 and the security door 105, at a location near the lock assembly 108. The unauthorized user can use the tool 110 to press a syringe plunger 101 and a syringe 132, so as to divert medicament.

Similarly, FIG. 1B also shows an unauthorized user diverting medicament by inserting a tool through a security door assembly, but at a different gap location. As seen in FIG. 1B, in a current security door assembly, the unauthorized user can also insert a tool 110 through a gap between the security frame assembly 102 and the security door 105, at a location at the front corner 107. As such, the unauthorized user can use the tool 110 to press a syringe actuator 103, so as to divert medicament.

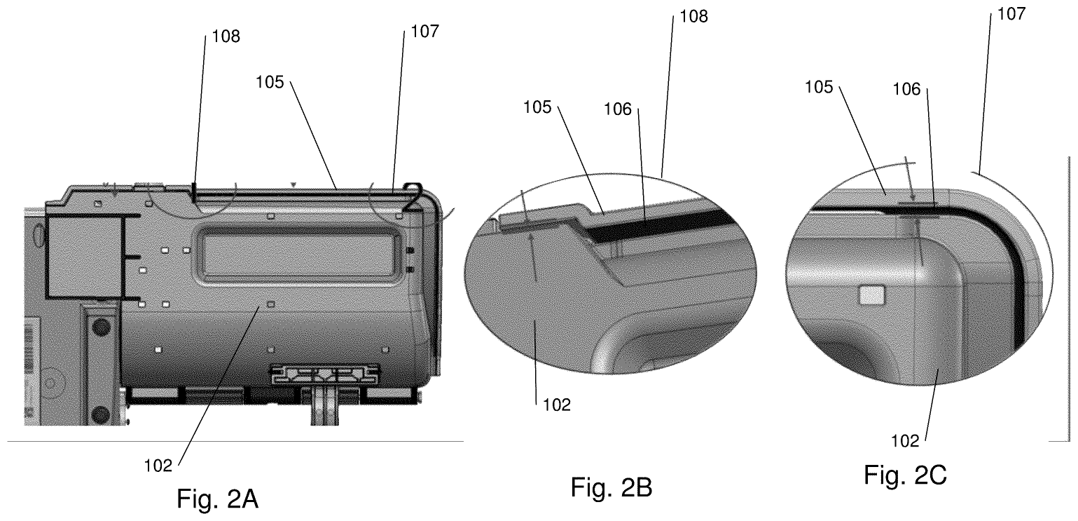

Relatedly, FIGS. 2A-C illustrate bottom views of a current security door assembly. FIG. 2A shows a bottom view of the current security door assembly when the security door 105 is placed on and closed on the security frame assembly 102. FIG. 2B shows a close-up view of the gap 106 between the security frame assembly 102 and the security door 105, at a location near the lock assembly 108 and FIG. 2C shows a close-up view of the gap 106 between the security frame assembly 102 and the security door 105, at a location at the front corner 107. As previously noted, unauthorized users can insert tools through the gaps 106 to divert medicament.

### Drug Diversion Guard

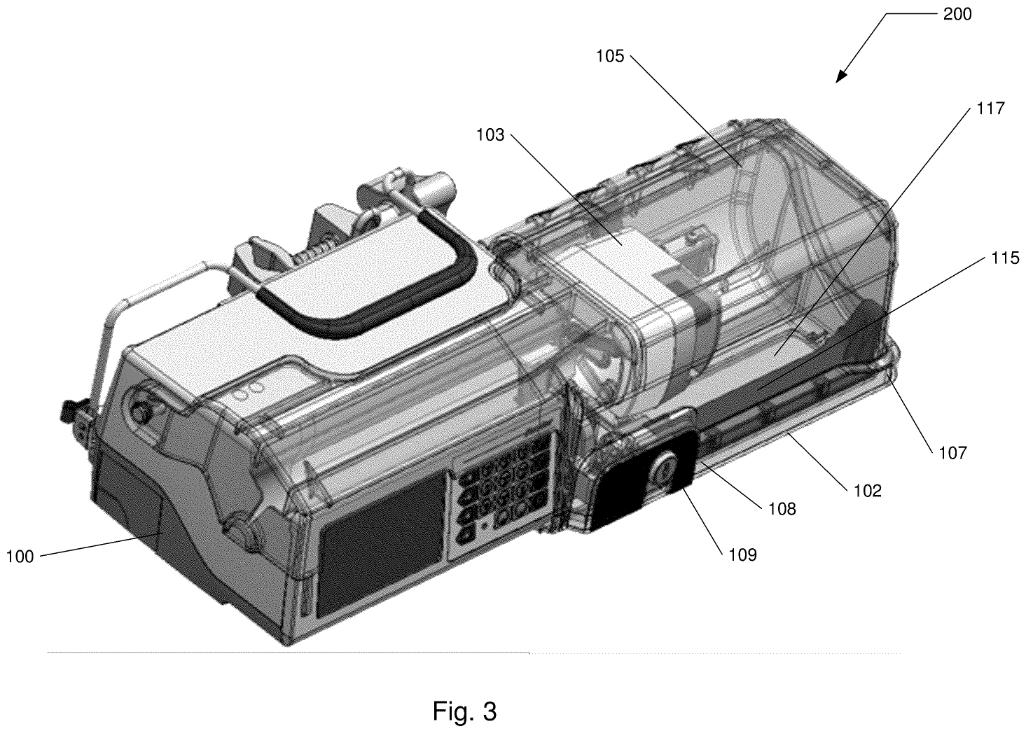

FIG. 3 illustrates a perspective view of an infusion pump utilizing an infusion pump drug diversion guard 200. In an embodiment, the security door 105 may be arranged with the security frame assembly 102 to create an enclosure for the pump 100. To achieve this, in an embodiment, the security door 105 and the security frame assembly 102 both include mating portions, which interlock to selectively couple the security door 105 and the security frame assembly 102 while still allowing the security door 105 to pivot about the security frame assembly 102. The resulting enclosure of the pump 100 completely encloses medicament-administering components of the pump 100, like a syringe actuator 103, making them inaccessible to unauthorized users. In an embodiment, when the security door 105 is unlocked and partially disengaged from the security frame assembly 102, the pump 100 and medicament-administering components of the pump 100, like a syringe actuator 103, are accessible to users. Overall, this means the security door 105 has an open and closed configuration, in terms of its pivoting relationship relative to the security frame assembly 102.

The security frame assembly 102 may include a lock dead bolt assembly 109 that is configured to engage with the security door 105 to ensure the security door 105 is retained in a locked position unless opened with the proper key. The lock dead bolt assembly 109 of the security door 105 and security frame assembly 102 ensures a physical key is needed to lock and unlock the pump 100 when delivering a medicament. In an embodiment, the lock dead bolt assembly 109 has been selected to be of a strong and robust material so as to minimize the possibility of an unauthorized user being able to break the lock dead bolt assembly 109 open. The security door 105 and the security frame assembly 102 can be integrated with the pump 100 and fastened to the pump 100 using fasteners (not shown) inaccessible for tampering. Once the security door 102 is closed and locked, the fasteners become inaccessible to the unauthorized user to prevent tampering with the security door 105, to access the syringe 132, and, therefore, the drug (not shown).

As seen in FIG. 3, an infusion pump drug diversion guard 115 is coupled to the security frame assembly 102. The infusion pump drug diversion guard 115 is selectively coupled to the security frame assembly 102 at the bottom 117 of the security frame assembly 102 between the security frame assembly 102 and the security door 105. Specifically, the infusion pump drug diversion guard 115 is located between the security frame assembly 102 and the security door 105 such that when the security door 105 is in a closed configuration, the infusion pump drug diversion guard 115 blocks the aforementioned gaps 106 (see FIGS. 2A-C) at the locations near the location near the lock assembly 108 and the location at the front corner 107. To achieve this objective, the infusion pump drug diversion guard 115 is shaped to complement the shape of the bottom 117 of the security frame assembly 102 between the security frame assembly 102 and the security door 105, such that it ‘fits’ into place.

It should be noted that the infusion pump drug diversion guard 115 is made of a strong and robust material so as to minimize the possibility of an unauthorized user being able to break the infusion pump drug diversion guard 115 by using a tool. Relatedly, the infusion pump drug diversion guard 115 is strongly coupled to the bottom 117 of the security frame assembly 102 such that an unauthorized user cannot, similarly, used a tool to uncouple the infusion pump drug diversion guard 115 from the security frame assembly 102 so as to divert medicament.

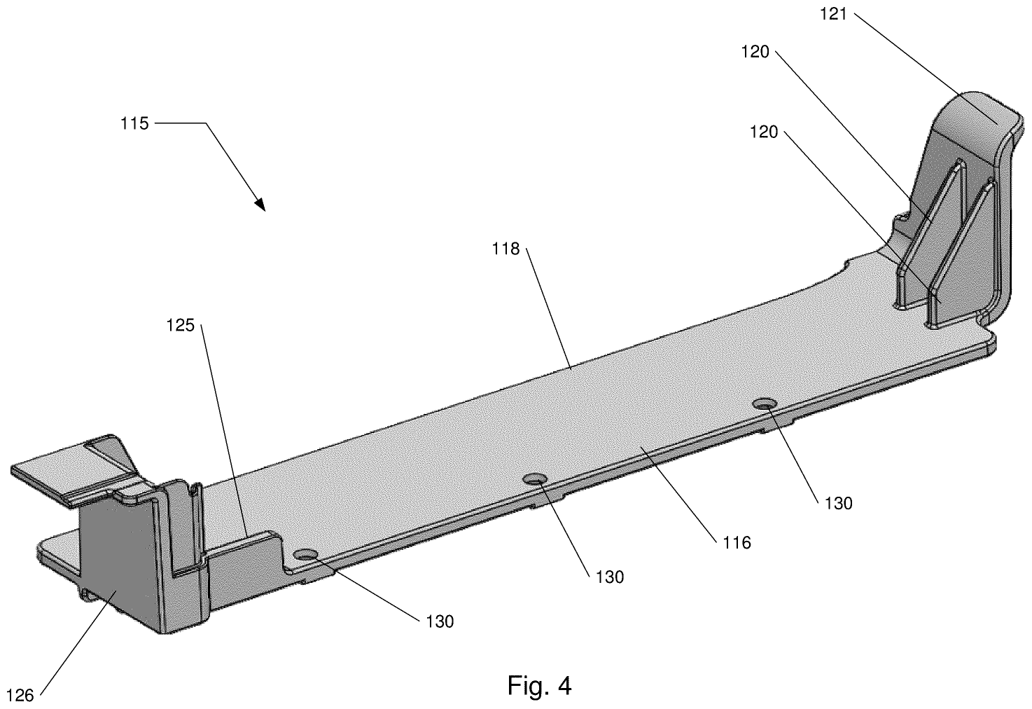

FIG. 4 illustrates a perspective view of an infusion pump drug diversion guard 115. As seen in FIG. 4, the infusion pump drug diversion guard 115 has a first end 126 opposite a second end 121, as well as a back end 118 opposite a front end 116. In an embodiment, the infusion pump drug diversion guard 115 includes coupling sections 130 which interact with coupling means (not shown), such as fasteners, to couple the infusion pump drug diversion guard 115 to a security door 105. The front end 116 is shaped to align with the front-most edge of the security frame assembly 102 such that a security door 105 can pivot to a closed configuration, as described above, when the infusion pump drug diversion guard 115 is coupled to the security door 105.

Notably, the first end 126 of the infusion pump drug diversion guard 115 is shaped to align with the security frame assembly 102 at the front-most edge near the lock dead bolt assembly 109, this position being described as the location near the lock assembly 108 in FIG. 3. The geometry of the first end 126 includes a blocking area 125. The dimensions of the blocking area 125 are configured to fully cover the gap 106 between the security frame assembly 102 and the security door 105, at a location near the lock assembly 108 from FIG. 3. The dimensions and geometry of the blocking area 125 can vary in alternate embodiments. Additionally, alternated embodiments can include multiple blocking areas 125.

The second end 121 of the infusion pump drug diversion guard 115 is shaped to align with the security frame assembly 102 at the front-most edge corner opposite the lock dead bolt assembly 109, this position being described as the location at the front corner 107 in FIG. 3. The geometry of the second end 121 includes a plurality of ribs 120. The geometry and dimensions of the ribs 120 are configured to fully cover the gap 106 between the security frame assembly 102 and the security door 105, at the location near the front corner 107 from FIG. 3.

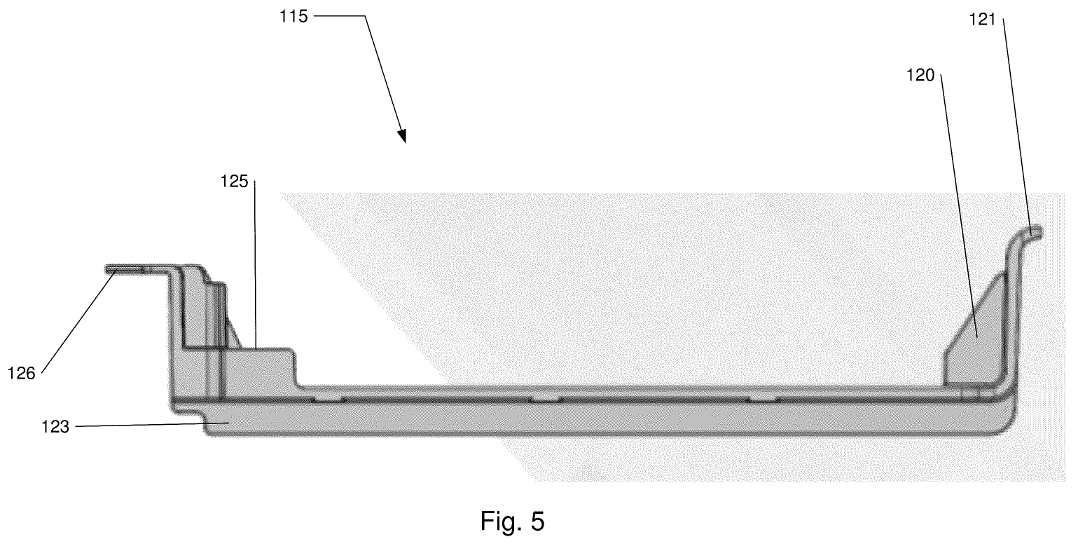

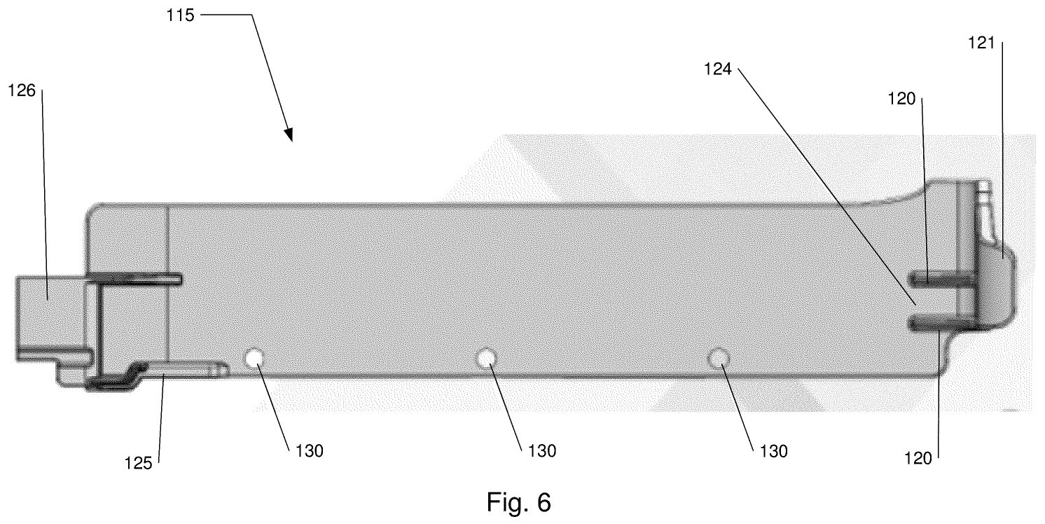

Further, FIG. 5 illustrates a front view of an infusion pump drug diversion guard 115. As seen in FIG. 5, the infusion pump drug diversion guard 115 may stand at a height 123. The height 123 can vary by embodiments such that the blocking area 125 and ribs 120 cover the afore mentioned gaps 106. FIG. 6 illustrates a top view of an infusion pump drug diversion guard. As seen in FIG. 6, there is a distance 124 between the ribs 120. The distance 124 between the ribs 120 can vary by embodiments such that the ribs 120 cover the afore mentioned gaps 106.

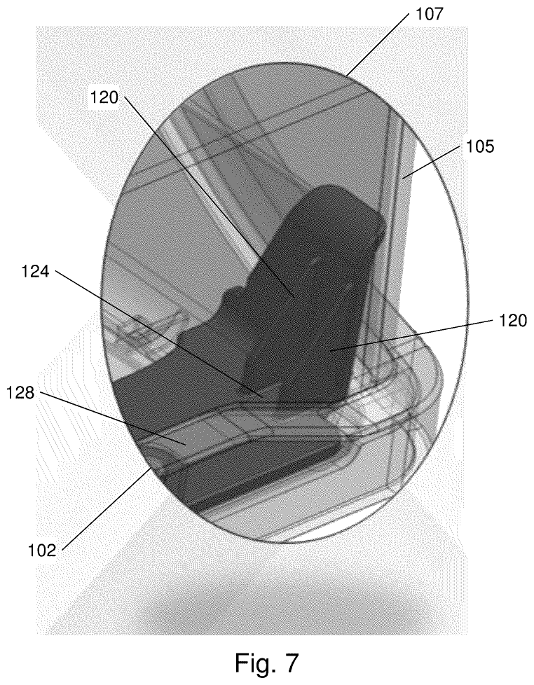

FIG. 7 illustrates close-up perspective views of a corner of a security door assembly on an infusion pump with an infusion pump diversion guard installed. As seen in FIG. 7, the infusion pump diversion guard 115 can include a corner lip 128. The corner lip 128 is shaped such that it mates with the security frame assembly 102. The geometry and dimensions of the corner lip 128 are configured to restrict tool access to the pump 100, to divert medicament. Together, the ribs 120 and the corner lip 128 fully cover the gap 106 between the security frame assembly 102 and the security door 105, at the location near the front corner 107. In turn, an unauthorized user is unable to use a tool to access the pump 100 and divert medicament by manipulating components such as a syringe plunger 101.

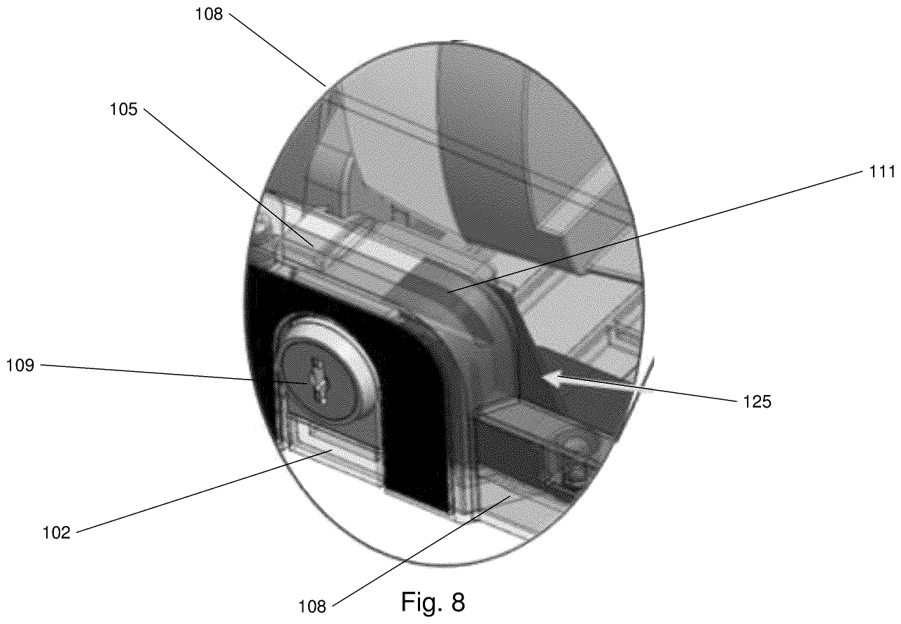

FIG. 8 illustrates a close-up perspective view of a lock area of a security door assembly on an infusion pump with an infusion pump diversion guard installed. As seen in FIG. 8, the infusion pump diversion guard 115 can include deadbolt lip 111 that is shaped to align flush with the lock dead bolt assembly 109 of the security door 105 and security frame assembly 102. The deadbolt lip 111 is shaped such that it mates with the security frame assembly 102. The geometry and dimensions of the deadbolt lip 111 are configured to restrict tool access to the pump 100, so as to divert medicament. Together, the blocking area 125 and the deadbolt lip 111 fully cover the gap 106 between the security frame assembly 102 and the security door 105, at the location near the lock assembly 108. In turn, an unauthorized user is unable to use a tool to access the pump 100 and divert medicament by manipulating components such as a syringe plunger 101.

It should be noted that in some embodiments, the security door 105 may include a variety of ribs, grooves and/or contours on the inside. The ribs, grooves and/or contours may be provided on the interior surface of the security door 105 to further minimize the possibility of an unauthorized user inserting some object while the security door 105 is closed and already locked. This results in a more robust security system, which further discourages unauthorized users from accessing the pump 100 for the purpose of drug diversion or other unauthorized tampering activities.

In an embodiment, the security door 105 may include a plurality of sensors. The sensors may be configured to detect medicament diversion. Additionally, the infusion pump diversion guard 115 may also include a plurality of sensors configured to detect unauthorized access to the pump 100. Also, in an embodiment, a plurality of sensors can be placed about the pump 100 in select locations to detect various conditions of the pump and/or security components. In response to the sensors receiving specific inputs, the pump 100 may initiate various alarm sequences. In turn, if an unauthorized user is still able to insert a tool into the pump so as to divert medicament while the security door 105 is locked with a loaded syringe, then as a further measure of security, additional alarms may be triggered.

It should be understood that various changes and modifications to the example embodiments described herein will be apparent to those skilled in the art. Such changes and modifications can be made without departing from the spirit and scope of the present subject matter and without diminishing its intended advantages. It is therefore intended that such changes and modifications be covered by the appended claims. Also, it should be appreciated that the features of the dependent claims may be embodied in the systems, methods, and apparatus of each of the independent claims.

Many modifications to and other embodiments of the invention set forth herein will come to mind to one skilled in the art to which these inventions pertain, once having the benefit of the teachings in the foregoing descriptions and associated drawings. Therefore, it is understood that the inventions are not limited to the specific embodiments disclosed, and that modifications and other embodiments are intended to be included within the scope of the appended claims. Although specific terms are employed herein, they are used in a generic and descriptive sense only and not for purpose of limitation.

The invention is claimed as follows:

1. A patient-controlled analgesia pump comprising:
a pump body;
a syringe holder;
a security door;
a security frame assembly; and
a diversion guard.

2. The pump of claim 1, wherein the pump body is configured to couple to the security frame assembly, and wherein the security door is configured to interlock with the pump body and security frame assembly such that the security door pivots relative to the security frame assembly.

3. The pump of claim 2, wherein the security door assembly can be oriented to a first position where the security door assembly pivots to a closed position, and wherein the security door assembly can be oriented to a second position where the security door assembly pivots to an open position.

4. The pump of claim 3, wherein the syringe holder is not accessible when the security door is in a closed position.

5. The pump of claim 3, wherein the syringe holder is accessible when the security door is in an open position.

6. The pump of claim 1, wherein the diversion guard is configured to selectively couple to the security frame assembly.

7. The pump of claim 6, wherein when the diversion guard is selectively coupled to the security frame assembly and the security door is in a closed position, the diversion guard covers a space between the security door and the security frame assembly.

8. The pump of claim 1, wherein the diversion guard is comprised of a strong and robust material, such that the diversion guard is breakproof.

9. The pump of claim 3, wherein the security frame assembly includes a deadbolt lock assembly configured to interact with a complimentary deadbolt receiver on the security door.

10. The pump of claim 9, wherein the deadbolt lock assembly and complimentary deadbolt receiver can be selectively locked together.

11. The pump of claim 1, wherein one of the security door or the security frame assembly further includes at least one sensor.

12. The pump of claim 11, wherein the at least one sensor is configured to detect the presence of at least one foreign object on the syringe holder when the security door is in a closed position.

13. A pump security system comprising:
a patient-controlled analgesia pump, wherein the patient-controlled analgesia pump comprises a syringe holder;
a security door;
a security frame assembly; and
a diversion guard.

14. The system of claim 13, wherein the security door and security frame assembly are configured to enclose the patient-controlled analgesia pump, and wherein the security door interlocks with the patient-controlled analgesia pump and security frame assembly such that the security door pivots relative to the security frame assembly.

15. The system of claim 14, wherein the security door assembly can be oriented to a first position where the security door assembly pivots to a closed position, and wherein the security door assembly can be oriented to a second position where the security door assembly pivots to an open position.

16. The system of claim 15, wherein the diversion guard is configured to selectively couple to the security frame assembly, and wherein when the diversion guard is selectively coupled to the security frame assembly and the security door is in a closed position, the diversion guard covers a space between the security door and the security frame assembly.

17. A diversion guard for a patient-controlled analgesia pump assembly, wherein the diversion guard selectively couples to the patient-controlled analgesia pump assembly.

18. The diversion guard of claim 17, wherein the diversion guard is comprised of a strong and robust material, such that the diversion guard is breakproof.

19. The diversion guard of claim 17, wherein the diversion guard is configured to cover a space between a security door and a security frame assembly when the patient-controlled analgesia pump assembly is in a closed configuration.

20. The diversion guard of claim 19, wherein when the diversion guard covers the space between a security door and a security frame assembly when the patient-controlled analgesia pump assembly is in a closed configuration, an unauthorized user cannot access a syringe holder in the patient-controlled analgesia pump assembly.
